(19) United States
(12) Patent Application Publication
(10) Pub. No.: US 2002/0193323 A1
(43) Pub. Date: Dec. 19, 2002

(54) Compositions and methods for promoting a healthy cardiovascular system and enhancing blood flow

(76) Inventor: Inna Yegorova, Northridge, CA (US)

Correspondence Address: SIERRA PATENT GROUP, LTD., P O BOX 6149, STATELINE, NV, 89449, US

(21) Appl. No.: 10/098,748

(22) Filed: Mar. 15, 2002

Related U.S. Application Data
(62) Division of application No. 09/717,274, filed on Nov. 22, 2000.

(51) Int. Cl. A61K 31/704; A61K 31/198; A61K 35/78; A61K 31/19

(52) U.S. Cl. 514/33; 424/195.17; 424/757; 424/754; 514/54; 514/185; 514/474; 514/561; 514/574; 514/458; 514/440

(57) ABSTRACT

Compositions and methods for promoting a healthy cardiovascular system and enhancing healthy blood flow by gently removing toxic buildup from the arterial walls and dilating and strengthening the arterial walls, in a mammal. The compositions comprise calcium; magnesium; selenium; manganese; zinc; potassium; vitamin E; vitamin A; alpha-lipoic acid; Allium sativum extract; Afecicago satiia extract; Chondrus crispus extract; L-cysteine; L-glutamic acid; glycine; and glutathione.

### FIELD OF THE INVENTION

The present invention relates to compositions and methods for promoting a healthy cardiovascular system and enhancing healthy blood flow by, e.g., gently removing toxic buildup from the arterial walls and dilating and strengthening the arterial walls.

### BACKGROUND OF THE INVENTION

Cardiovascular disease (CVD) is the leading cause of death in the industrialized countries. According to the American Heart Association, in 1997 alone, 59.7 million 1 g Americans were estimated to have one or more forms of cardiovascular disease including high blood pressure, coronary heart disease, stroke and rheumatic heart disease. Of those, 953,110 Americans died of some form of CVD, attributing 41.2% of all deaths to CVD. Coronary heart disease (CHD) by itself can be attributed to causing about half of all deaths associated with cardiovascular diseases (49%). The primary cause of CHD is artheriosclerosis, a degenerative change in the arteries whereby the arterial walls thicken and lose elasticity. One key process of artheriosclerosis is the accumulation of lipids resulting in distribution of atheromatous plaque. As plaque accumulates in the inner artery wall, the restricted artery is weakened, bulging with cholesterol and toxic deposits. Eventually, the plaque blocks the arteries and interrupts blood flow to the organs they supply.

It is now well established that vascular blockage and cardiovascular disorders including myocardial infarction, coronary heart disease, hypertension and hypotension, cerebrovascular disorders including stroke, cerebral thrombosis and memory loss due to stroke; peripheral vascular disease and intestinal infarction are caused by blockage of arteries and arterioles by atherosclerotic plaque. The production of atherosclerotic plaque formation is multi-factorial in its production. Hypercholesterolemia, especially elevated levels of low-density lipoprotein cholesterol (LDL) is an important risk factor for atherosclerosis and arteriosclerosis and associated diseases.

Current therapeutic interventions in cardiovascular diseases range from balloon angioplasty to cardiac transplantation. Balloon angioplasty involves the dilation of an artery by a percutaneously inserted balloon catheter. The long-term success of angioplasty, however, is limited. Within the first 4 to 6 months after angioplasty, approximately 30 to 50% of patients develop a re-narrowing of the widened artery (Gottsauner-Wolf et al., 19 CLIN. CARDIOL. 347 (1996)). More drastic measures involve vascular replacement surgery or coronary artery bypass graft surgery where the occluded artery is either replaced or bypassed to restore blood circulation. In cases of severe intractable heart failures, cardiac transplantation is attempted. Major complications associated with heart transplants include organ rejection and infection. Despite technological advances, the current 5-year survival rate for the procedure is merely in the sixtieth percentile (O'Connell et al., 86(3) CIRCULATION 1061-79 (1992)). Given the lack of success in therapeutic measures and the cost of current therapeutic procedures (angioplasty costing approximately $21,000 and bypass surgery costing approximately $44,000 per operation), there is a need to redirect t: efforts towards preventive medicine.

It is now generally known that there are three forms of cholesterol: very low-density u lipoprotein (VLDL), low-density lipoprotein (LDL) and high-density lipoprotein (HDL). Arterial wall cholesterol, and therefore atherosclerotic plaque, consists almost exclusively of LDL (Brown and Goldstein, 52 ANN. REV. BIOCHEM. 223 (1983)). Overwhelming evidence shows that LDL cholesterol becomes harmful only in its oxidized form known as oxysterol (Schwartz et al., 71 AM. J. CARDIOL. 9B-14B (1993); Jialal and Grundy, 669 ANN. N.Y. ACAD. SCI. 237-48 (1992)). HDL on the other hand, has been found to be inversely associated with coronary artery disease (Rader, 83(9B) AM. J. CARDIOL. 22F-4F (1999)) It has been determined that for every 1 percent increase in the HDL cholesterol level, the risk of having a coronary event is decreased 3 percent. There are now two general approaches towards preventing CVD. The first is to lower LDL cholesterol levels and/or increase HDL cholesterol levels and the other is to reduce levels of oxidized cholesterol.

Several studies have demonstrated that lowering LDL cholesterol levels reduces death from heart disease. The Scandinavian Simvastatin Survival Study followed 4,444 men and women with a history of angina or heart attack over 5.4 years (344 LANCET 1383-1389 (1994)). The study showed that simvastatin, a cholesterol lowering drug, was effective at lowering LDL and decreasing the total deaths and need for bypass and angioplasty surgery. The Cholesterol and Recurrent Events Trial demonstrated that pravastatin, another cholesterol lowering drug, was effective at lowering LDL cholesterol by 28%, heart attacks by 25%, and strokes by 28%. The study involved 4,158 men and women with a recent history of heart attack (Sacks et al., 335 N. ENGL. J. MED. 1001-1009 (1996)).

A host of LDL cholesterol lowering drugs is currently on the market. The most widely used lipid-lowering drugs include simvastatin (Zocor®)), pravastatin (Pravachol®), lovastatin (Mevacor®), fluvastatin (Lescol®), atorvastatin (Lipitor®), and cerivastatin (Baycol®), which make up the group of HMG-CoA reductase inhibitors known as statins. The statins inhibit one of the enzymes responsible for manufacturing VLDL in the liver (HMG-CoA reductase). In response to a lower level of VLDL, the liver removes LDL from the bloodstream to compensate for the loss of VLDL, thereby reducing LDL cholesterol levels in the blood. Statins have also been found to increase HDL levels in some patients. Although effective, the statins are associated with several side effects including reversible liver enzyme elevations, gastrointestinal upset, headache, dizziness, mild skin rashes, muscle pain and muscle inflammation at high does. Moreover, serious liver toxicity is possible and thus liver function should be checked regularly. Side effects notwithstanding, recent coronary angiography trials have revealed that even if LDL cholesterol can be lowered below 100 mg/dl using cholesterol lowering drugs, atherosclerosis progression is arrested in only 50% to 60% of patients. Alternative cholesterol lowering drugs include: (1) fibrates, gemfibrozil (Lopid®) and clofibrate (Atromid-D®), which activates the enzyme lipoprotein lipase, resulting in a lowering of triglycerides and possibly VLDL, and (2) bile acid sequestrants, better known as resins, cholestyramine (Questran®) and colestipol (Colestid®), which bind bile acids in the intestines and carries them out. The liver requires cholesterol to make more bile acids and therefore removes LDL from the blood for this function. Both fibrates and resins have not found widespread use because the former is associated with hepatitis and a two-fold increased risk of gallstones and the later with gastrointestinal discomfort and an increase in triglycerides, another CHD risk factor. An analysis of several studies even showed a slight increase in overall deaths due to the use of fibrates (Farmer and Gotto, 11(5) DRUG SAF 301-9 (1994); Grundy 70(21) AM J CARDIOL 271-321 (1992); 40(1030) MED. LETRER DRUGS THER. 68-9 (1998)).

By the 1980's, it was recognized that HDL may prevent the development of CHD (Frohlich and Pritchard, 22 CLIN. BIOCHEM. 417-33 (1989)). Factors such as smoking, obesity, hypertriglyceridemia, genetic factors and lack of exercise are major causes of reduced serum HDL. HDL cholesterol lipoproteins move excess cholesterol from the extrahepatic organs to the liver for excretion (Dietschy, 65 AM. J. CLINICAL NUTRITION 1581S-9S(1997)). There is evidence that virtually everybody tissue is capable of at least some cholesterol synthesis from the precursor acetyl-coenzyme A (CoA). Every day, HDL carries back to the liver an amount of cholesterol equal to the amount synthesized and taken up as LDL by all extrahepatic organs except endocrine glands. There is a second LDL transport process that is receptor independent. Id. Removal of free cholesterol from arterial wall cells may be an important mechanism by which HDL plays an anti-atherogenic role (Goodman et al., GOODMAN AND GILMAN'S THE PHARMACOLOGICAL BASIS OF THERAPEUTICS 878 (J. G. Hardman and L. E. Limbird eds., 9th ed., McGraw-Hill, 1941)(1996)). Gemfibrozil and niacin or preferably a combination of the two, has been shown to be effective for increasing HDL cholesterol levels (Zema, 35 J. AM. COLL. CARDIOL. 640-6 (2000)). However, side effects associated with niacin (Guyton, 82 AM. J. CARDIOL. 18U-23U (1998)) and gemfibrozil (as described above) has overshadowed their usefulness.

It has now been generally accepted that LDL cholesterol becomes harmful only in its oxidized form (Schwartz et al., supra; Jialal and Grundy, supra). Native LDL consits of phospholipids, triglycerides, cholesterol, both free and as an ester, fatty acids (50% of which is polyunsaturated), proteins and lipophilic antioxidants that protect the polyunsturated fatty acids (PUFA) in cholesterol against free radical attack and oxidation. The first step to the oxidation of cholesterol is the production of free radicals generally induced by oxidative stress. These radicals act not only to deplete lipids of their natural antioxidants, such as vitamin E and carotinoids, but are also highly reactive against proteins, DNA, PUFA and lipids. Once the natural antioxidants are depleted, the free radicals move to oxidize unprotected LDL. The oxidized cholesterol molecule is recognized by scavenger receptors and internalized by macrophages in the form of lipid loaden foam cells, the first step to formation of artherosclerotic plaque (Esterbauer et al., 38 ADV. PHARMACOL. 425-56 (1997); Esterbauer, 2 NUTR. METAB. CARDIOVASC. DIS. 55-7 (1992)). Oxidative stress may occur when formation of reactive oxygen species increases, scavenging of reactive oxygen species or repair of oxidatively damaged macromolecules decreases, or both. Thus, factors such as exposure to environmental pollutants and pesticides can instigate the generation of oxysterols internally.

More recent efforts towards aniatherogenic drugs have been directed at compounds with antioxidative properties. Amlodipine, a calcium antagonist, was determined to normalize elevated levels of oxidized LDL cholesterol without reducing elevated total plasma cholesterol levels. Initial results indicated that atherosclerosis progression was suppressed in monkeys who had been fed an atherogenic diet (Kramsch, 62(Suppl 2) INT. J. CAPDIOL. S119-24 (1997)). Monatepil, an alpha 1-adrenoceptor-blocking drug with antilipid peroxidation activities was also found to reduce plasma lipid levels (Miyazaki, 7(10 Pt 2) AM. J. HYPERTENS. 131S-40S (1994)). Substituted phenols and thiophenols have been documented as antioxidant chemicals for inhibiting the peroxidation of LDL cholesterol as well (U.S. Pat. No. 6,114,572).

It has been shown that oxidation of LDL may be stimulated by the presence of certain metals such as Cu²⁺ (Esterbauer et al., 3(2) CHEM. RES. TOXICOL. 78-92 (1990)). In fact, atherogenicity of homocysteine is related to copper-dependent interactions (Mansoor et al., 46(3) CLIN. CHEM. 385-91 (2000)). Iron has also been implicated in the process of LDL oxidation by macrophages (Karten et al., 40(7) J. LIPID. RES. 1240-53 (1999)). Both copper and iron are transitional metals known to catalyze formation of more reactive hydroxyl radicals (Olanow, 16 TRENDS. NEUROSCI. 439-44 (1993)), the cause of LDL cholesterol oxidation discussed above. Recent studies with polyphenols, found in vegetable tannins, show that they exert their effects not only as antioxidant agents but also as strong chelators to heavy metals (Haslam, 59 J. NAT. PROD. 205-215 (1996)). Chelation therapy to remove heavy metals from the blood has become another alternative to the prevention and treatment of CVD. Recent literature on chelators include Lovejoy et al., 6 METAL IONS IN BIOLOGY AND MEDICINE 221-3 (2000); Richardson et al., 86 BLOOD 4295 (1995); Defraigne et al., 8(5) ANN. VASC. SURG. 457-67 (1994); and Menasche et al., 100(1) J. THORAC. CARDIOVASC. SURG. 13-21 (1990). Synthetic chelators such as exochelin (U.S. Pat. No. 5,721,209) and desferrioxamine (U.S. Pat. Nos. 5,663,201, 5,739,167, 5,811,127, and 5,869,446) have been used for inhibition of iron-mediated oxidant injury. However, notable drawbacks include limited cellular penetration.

Although preventive measures are currently available, a more multi-dimensional approach is needed. These measures preferably include dietary supplementation that focuses on the cardiovascular system as a whole rather than the individual conditions that affect the system. A better and natural method for promoting a healthy cardiovascular system and enhancing blood flow that is readily available and cost-efficient is needed. A primary objective of the present invention is the administration of a composition that will promote a healthy cardiovascular system and enhance blood flow. One aspect of the present invention is the reduction of toxins from the arterial wall. Another aspect of the present invention is the dilation of the-arterial wall. Yet another aspect of the present invention is the strengthening of the arterial wall.

### SUMMARY OF THE INVENTION

The present invention relates to compositions and methods for promoting a healthy cardiovascular system and enhancing blood flow. These compositions preferably comprise one or more of: calcium; magnesium; selenium; manganese; zinc; potassium; vitamin E; vitamin A; alpha-lipoic acid; Allium sativum extract; Medicago saliva extract; Chondrus crispus extract; L-cysteine; L-glutamic acid; glycine; and glutathione.

In one embodiment of the present invention, the composition may comprise calcium in an amount ranging from 24 mg to 36 mg, magnesium in an amount ranging from 24 mg to 36 mg, selenium in an amount ranging from 16 mcg to 24 mcg, manganese in an amount ranging from 0.3 mg to 0.7 mg, zinc in an amount ranging from 4.0 mg to 6.0 mg, potassium in an amount ranging from 24 mg to 36 mg, vitamin E in an amount ranging from 16 IU to 24 is IU, vitamin A in an amount ranging from 2000 IU to 3000 RU, alpha-lipoic acid in an amount ranging from 16 mg to 24 mg, Allium sativum extract in an amount ranging from 16 mg to 24 mg, Medicago sativa extract in an amount ranging from 16 mg to 24 mg, Chondrus crispus extract in an amount ranging from 24 mg to 36 mg; L-cysteine in an amount ranging from 16 mg to 24 mg, L-glutamic acid in an amount ranging from 16 mg to 24 mg, glycine in an amount ranging from 16 mg to 24 mg, and glutathione in an amount ranging from 12 mg to 18 mg.

In accordance with the present invention, methods and compositions are provided for use in treating atherosclerosis and its associated diseases including cardiovascular disorders, cerebrovascular disorders, peripheral vascular disorders, and intestinal vascular disorders. The methods and compositions of the present invention are particularly advantageous in that they may be used to both significantly lower plasma cholesterol levels and substantially arrest, reverse and/or cure the arterial plaque deposition and degenerative vascular wall changes associated with atherosclerosis.

The compositions of the present invention can be administered prophylactically, so as to inhibit atherogenesis or restenosis, or therapeutically after CVD or atherogenesis has been diagnosed or initiated. Thus, for example, a patient who is to undergo balloon angioplasty can have a regimen of the composition administered substantially prior to the balloon angioplasty, preferably at least about a week or substantially longer. Alternatively, in a patient where atherogenesis is suspected, the administration the composition can begin at any time. Administration may be accomplished in any manner known to those skilled in the art, including peroral, liposomal, inhalation, sublingual, rectal (e.g., suppositories), or through an oral spray or dermal patch.

Methods are provided for lowering blood pressure, modulating the production of LDL-cholesterol and reducing the oxidation of LDL into lipid peroxides. As a prophylactic or treatment for atherosclerotic susceptible hosts, the composition is chronically administered at an effective dosage. For restenosis, the agent may be administered for a limited period since this pathological process generally abates 3-6 months after the vascular injury (i.e., angioplasty or atherectomy).

In another aspect, the invention is a method of altering the concentration of cholesterol constituents in the blood of a human, to preferably reduce the risk of atherosclerosis and vascular disease, where the composition is administered to a human in an fir, amount effective to increase the concentration of HDL-cholesterol in the blood of the human. U Reducing cholesterol levels with the administration of this composition can also prevent other plaque formation and other types of atherosclerotic disease such as the cereberovascular complications of carotid artery plaques, peripheral vascular disease and claudication, and intestinal vascular blockage and infarction.

In another embodiment of the present invention, the composition may be administered to a mammal. Preferably, the mammal is a human. The preparations may be in solid form, for instance, in capsule, powder or granule, or tablet form. Alternatively, the compositions may be dispersed into a suitable liquid. The composition may also be administered orally, preferably three to five times daily.

Another embodiment of the invention involves administering the compositions of the present invention to a human as a material dietary supplement. In yet another embodiment of the invention the composition is administered to a human as a pharmaceutical composition.

### DETAILED DESCRIPTION OF THE INVENTION

The present invention relates to the administration of compositions and methods for promoting a healthy cardiovascular system and enhancing blood flow. It is understood that the present invention is not limited to the particular methodology, protocols, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise.

In accordance with the present invention, cardiovascular health may be determined by four parameters: blood pressure; HDL cholesterol level; LDL cholesterol level; and lipid peroxide level. Blood pressure results from a combined force of blood pumping into the arteries and through the circulatory system and the force created as the arteries resist blood flow. In a healthy individual, blood pressure is less than 140 mm Hg over 90 mm Hg (see Table 1). The higher number, systolic pressure, represents the pressure while the heart is beating and the lower number, diastolic pressure, is the pressure when the heart is resting between beats (National High Blood Pressure Education Program/National Institutes of B Health. The fifth report of the Joint National Committee on Detection, Evaluation, and Treatment of High Blood Pressure. NIH publ. no. 93-1088 (1993)).

TABLE 1
American Heart Association recommended blood pressure levels.

| Blood | | | |
|---|---|---|---|
| Pressure | Systolic | | Diastolic |
| Category | (mm Hg) | | (mm Hg) |
| Optimal | less than | and | less than |
| | 120 | | 80 |
| Normal | less than | and | less than |
| | 130 | | 85 |
| High normal | 130-139 | or | 85-89 |
| High | 140-159 | or | 90-99 |
| Stage 1 | | | |
| (mild) | | | |
| High | 160-179 | or | 100-109 |
| Stage 2 | | | |
| (moderate) | | | |
| High | 180 or | or | 110 or |
| Stage 3 | higher | | higher |
| (severe) | | | |

LDL cholesterol levels are also very indicative of CHD risk. The National Cholesterol Education Program (NCEP) has classified optimal LDL cholesterol levels at <100 mg/dL (see Table 2) (269 JAMA 3015-23 (1993); 89 CIRCULATION 1329-445 (1994)).

TABLE 2
NCEP classification of Total and LDL-Cholesterol.

| | Total Cholesterol | LDL-Cholesterol |
|---|---|---|
| Classification | (mg/dL) | (mg/dL) |
| Optimal | <150 | <100 |
| Desirable | 150-199 | 100-129 |
| Mild Hypercholesterolemia | 200-239 | 130-159 |
| Moderate | 240-299 | 160-219 |
| Hypercholesterolemia | | |
| Severe Hypercholesterolemia | 300 | 220 |

It is recommended that persons with elevated total cholesterol concentrations above 0.240 mg/dL (6.2 mM/L) receive treatment and that those with borderline values between 200-239 mg/dL (5.2 to −6.2 mM/L) be further evaluated according to the presence of risk factors for coronary artery disease including the sex of the patient, post-menopausal status, a low plasma concentration of HDL cholesterol (below 35 mg/dL [0.9 mM/L]), positive family history, smoking, hypertension and diabetes mellitus (Expert Panel on Detection, Evaluation, and Treatment of High Blood Cholesterol in Adults, 269(23) J. AM. MEDICAL A. 3015-3023 (1993)). Other factors include obesity, hypertriglyceridemia, sedentary lifestyle, steroid use, β-adrenergic blocking agents, some diuretics and genetic factors (Frohlich and Pritchard, supra.)

Thus in a preferred embodiment of the invention, administering of the composition of the invention will maintain optimal total, LDL and HDL cholesterol levels. As previously, discussed, lipid peroxidation is an indication of oxidative stress and thus the presence of lipid peroxides have been linked to cardiovascular disease. In another preferred embodiment of the invention, administering of the composition of the invention will increase HDL cholesterol levels and reduce elevated LDL cholesterol levels and lipid peroxide levels.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references cited herein are incorporated by reference herein in their entirety.

### Definitions

Calcium, as described herein, includes the various forms of calcium available, including but not limited to, calcium orotate, calcium absorbate, calcium aspartate, calcium carbonate, calcium chelate, calcium citrate, calcium citrate malate, calcium gluconate, calcium lactate, dolomite, oyster shell calcium and tricalcium phosphate.

Magnesium, as described herein, includes the various forms of magnesium available, including but not limited to, magnesium succinate, magnesium chloride, magnesium citrate, magnesium fumarate, magnesium gluconate, magnesium malate, magnesium oxide and magnesium sulfate.

Selenium, as described herein, includes the various forms of selenium available, including but not limited to, selenomethionine, selenite, selenium dioxide and selenized yeast.

Manganese, as described herein, includes the various forms of manganese available, including but not limited to manganese ascorbate, manganese chloride, manganese gluconate, manganese picolinate and manganese sulfate.

Zinc, as described herein, includes the various forms of zinc available, including but not limited to, zinc orotate, chelated zinc, zinc citrate, zinc gluconate, zinc picolinate and zinc sulfate.

Potassium, as described herein, includes the various forms of potassium available, including but not limited to, potassium citrate, chelated potassium, potassium aspartate, potassium bicarbonate and potassium chloride.

Vitamin E, as described herein, includes the various forms of vitamin E available, including but not limited to, alpha-tocopherol, D-alpha-tocopherol, D-beta-tocopherol, D-delta-tocopherol, D-gamma-tocopherol, D-tocopherol, DL-alpha-tocopherol, DL-tocopherol, mixed tocopherol, tocopheryl acetate and tocopheryl succinate. One international unit (IU) of vitamin E is referred to as 1 mg of the synthetic form, racemic alpha-tocopheryl acetate. Thus, the natural form of d-alpha-tocopherol has a biopotency of vitamin E equal to 1.49 IU.

Vitamin A, as described herein, includes the various forms of vitamin A available, including but not limited to beta-carotene and retinol. One international unit (RU) of vitamin A activity has been defined as equal either to 0.30 μg of all-trans retinol or to 0.60 μg of all-trans β-carotene.

Alpha-lipoic acid, as described herein, includes the various forms of alpha-lipoic acid including but not limited to lipoic acid and thioctic acid.

Allium sativum, as described herein, will also be referred to as garlic. Allium sativum preferably contains 0.8% allicin in extract form.

Allicin, as described herein, is diallyl thiosulfinate.

Medicago sativa, as described herein, will also be referred to as alfalfa. Medicago sativa preferably contains triterpenoid saponins.

Triterpenoid saponins, as described herein, is understood widely as a mixture of different glycosides including medicagenic acid glycosides, hederagenin glycosides, zanhic acid glycosides and soyasapogenol B and its dehydro glycosides.

Chondrus crispus, as described herein, will also be referred to as Irish moss. Chondrus crispus preferably contains carrageenans.

Carrageenans, as decribed herein, is understood widely as a mixture of kappa carrageenans, iota carrageenans and lambda carrageenans.

Glutathione, as described herein, includes a tri-peptide comprising cysteine, glutamic acid and glycine.

The components of the compositions of the present invention may be divided into three main categories: minerals, antioxidants, and chelators. Any single component may exert its effects through one or more mechnanisms. For example, alpha-lipoic acid is a known chelator with antioxidant properties.

Minerals are inorganic elements that serve a variety of functions in the body. Minerals may function as cofactors in enzyme-catalyzed reactions, in the regulation of acid-base balance, in nerve conduction and muscle irritability, and as structural elements in the body (Champe and Harvey, LIPPINCOTT'S ILLUSTRATED REVIEWS: BIOCHEMISTRY 307 (1987)). Minerals are divided into two categonies: macrominerals and microminerals. A macromineral or bulk element is a mineral required by humans in a large amount, e.g., 100 mg/day or more (Mahan and Stump, KRAUSE's FOOD, NUTRITION, AND DIET THERAPY 112 (2000)). In contrast, a micromineral, also known as a trace element, is a mineral that is required by humans in quantities of a few milligrams or even micrograms per day. Id at 124. Macrominerals include calcium, phosphorous, magnesium, and potassium, and the microminerals include zinc, iodine, manganese, and selenium.

The use of antioxidant vitamins for the prevention of coronary heart disease is becoming more prevalent as the proportion of the population seeking alternative therapies is increasing (Eisenberg et al., 280(18) JAMA 1569-75 (1998)). As discussed supra, overwhelming evidence show that LDL cholesterol becomes harmful only in its oxidized form known as oxysterol (Schwartz et al., sitpra; Jialal and Grundy, sitprci). Excess free radicals present in plasma increase LDL oxidation (Diaz et al., 337 N. ENGL. J. MED. 408-16 (1997)) but can be inhibited by antioxidant vitamins. Animal studies have demonstrated that an increased intake of antioxidant nutrients may have a role in preventing coronary heart disease (Ulbricht et al., 338 LANCET 985-92 (1991)).

Factors, such as the presence of transition metals and exposure to environmental pollutants and pesticides, can also instigate the generation of oxysterols internally. Transition metals are especially well known to catalyze generation of free radicals (Olanow, supra) and have been linked to the oxidation of LDL cholesterol (Makjanic et al., 159 NUCL. INST. METH. PHYS. RES. SECT. B 356-60 (1999); Swain et al., 343 FEBS LETT. 49-52 (1994); Smith et al., 286 BIOCHEM. J. 901-5 (1992)). Chelation therapy has been shown to be safe and effective for treatment and prevention of atherosclerosis (Olszewer and Carter, A TEXTBOOK ON EDTA CHELATION THERAPY 197-211((E. M. Cranton ed., special issue, JOURNAL OF ADVANCEMENT IN MEDICINE 2(1-2), Human Sciences Press) (1989); McDonagh et al., A TEXTBOOK ON EDTA CHELATION THERAPY 155-6((E. M. Cranton ed.; special issue, JOURNAL OF ADVANCEMENT IN MEDICINE 2(1-2), Human Sciences Press) (1989)). In fact, it has been implied that chelation therapy is a necessary complement to antioxidant supplementation (Halliwell, 355 LANCET 1179-80 (2000)). Several paradoxical results from studies of antioxidant vitamins show that in the presence of transition-metal ions, antioxidant vitamins actually exert pro-oxidant effects (Kang et al., 28 FREE RADIC. RES. 93-107 (1998); Maiorino et al., 330 FEBS LETT. 174-6 (1993)).

Accordingly, one aspect of the present invention is the use of a combination of natural minerals, antioxidants and chelators to promote a healthy cardiovascular system and encourage healthy blood flow.

In a preferred embodiment, the compositions of the present invention may comprise magnesium in amounts preferably ranging from about 24 mg to about 36 mg. Magnesium is an essential nutrient that may be found in the form of magnesium succinate. It acts as a calcium channel blocker but also functions as an activator of many enzymes in the body. Together, magnesium and calcium work cooperatively to support and maintain heart function. Studies have shown that magnesium supplementation can reduce high blood pressure (Sanjuliani et al., 56 LNT. J. CARDIOL. 177-83 (1996); Witteman et al., 60 AM. J. CLIN. NUTR. 129-135 (1994)). U.S. Pat. No. 6,100,297 relates to the use of magnesium as an effective treatment for conditions caused by excessive oxidative stress. Accordingly, magnesium is an Important chelating agent that promotes absorption and metabolism of other minerals. It helps the coronary arteries to relax and open for greater blood flow. A preferred embodiment of the compositions of the present invention comprise about 30 mg magnesium.

The compositions of the present invention may comprise potassium, preferably in an amount in the range of about 24 mg to about 36 mg. Potassium is necessary for growth, helps in the contraction of muscles, and preserves alkalinity of the body's fluids. A review of 33 studies indicates that dietary potassium will help reduce high blood pressure (Whelton et al., ANN. EPIDEMIOL. 85-95 (1995)). Potassium may be found in the form of potassium citrate. A preferred embodiment of the compositions of the present invention may comprise about 30 mg of potassium.

In another preferred embodiment, the compositions of the present invention may comprise selenium in amounts preferably ranging from about 16 mcg to about 24 mcg. Selenium is a trace mineral involved in fat metabolism found in the form of selenomethionine. Dietary selenium is a potent antioxidant that can reduce susceptibility to lipid peroxidation by activating the glutathione system, as discussed infra (Hussein et al., 63(5) TRANSPLANTATION 679-85 (1997)). Selenomethionine is the most bioavailable form of selenium, with D-selenomethionine only one-fifth as bioavailable (Thomson et al., 36 AMER. J. CLIN. NUTR. 24-31 (1982)), and has been found to be superior to inorganic forms of t selenium. Selenium supplementation therefore preserves elasticity of the arterial walls by delaying the oxidation of polyunsaturated fatty acids that can cause solidification of tissue proteins. A preferred embodiment of the compositions of the present invention may comprise about 20 mcg selenium.

The compositions of the present invention may also comprise one or more of the amino acids L-cysteine, L-glutamic acid or glycine, preferably ranging from about 16 mg to about 24 mg of L-cysteine, L-glutamic acid and glycine each, and about 12 mg to about 18 mg of glutathione. L-Cysteine, L-glutamic acid and glycine form glutathione, an antioxidant and detoxifier important in many enzyme systems (Chanvitayapongs et al., 8 NEUROREPORT 1499-1502 (1997))., Antioxidant enzymes made from glutathione and selenium, including glutathione peroxidase (GSH-Px) and phospholipid hydroperoxide glutathione peroxidase (phGSH-Px), protect cells against oxidative stress. GSH-Px protects against oxidation and against environmental and metabolic toxins which cause peroxidation, while phGSH-Px protects membranes against peroxides already bound to membrane surfaces (Ursini et al., 839 BIOCHIM. BIOPHYS. ACTA 62-70 (1985)). In a recent study, dietary selenium was found to elevate glutathione peroxidase levels and to reduce glutathione content. Through the activation of the glutathione system, selenium supplementation reduced lipid peroxidation levels by 50%, and may thus be considered antiatherogenic (Hussein et al., supra) Cysteine, has become known as an important chelator. In sufficient levels, it will bind with metals and is know to promote the excretion of excess copper. A preferred embodiment of the compositions of the present invention may comprise 20 mg each of one or more of the amino acids L-cysteine, L-glutamic acid or glycine. In another preferred embodiment, the compositions comprise about 20 mg of glutathione.

The novel compositions of the present invention may also comprise calcium, preferably in an amount in the range of about 24 mg to about 36 mg. Calcium, the most abundant mineral in the body, plays a role in ion transport across cell membranes and is essential to teeth and bones. Calcium deficiency leads to increased blood pressure, while calcium supplementation has been shown to reduce total and LDL cholesterol and raise HDL cholesterol (Cappuccio et al., 142(9) AM. J. EPIDEMIOL. 935-45 (1995); Van Leer et al., 24(6) INT. J. EPIDEMIOL. 11 17-23 (1995); Bell et al., 152 ARCH. INTERN. MED. 2441-4 (1992)). Calcium is found in the forms of calcium orotate and calcium abscorbate. A preferred embodiment of the compositions of the present invention may comprise about 30 mg calcium.

The compositions of the present invention may comprise manganese, preferably in an amount in the range of about 0.3 to about 0.7 mg. Manganese is found in high concentrations in bone, pituitary, liver, pancreas, and gastrointestinal tissue. This trace mineral is a constituent of essential enzyme systems and has been implicated in an important role as part of the natural antioxidant enzyme superoxide dismutase. A preferred embodiment of the compositions of the present invention may comprise about 0.5 mg manganese.

Both the calcium and manganese of the present invention preferably may be in the form of ascorbates, better known as vitamin C. Vitamin C is a powerful water-soluble antioxidant which complements vitamin E, a lipophilic antioxidant vitamin. Importantly, vitamin C increases the absorption of selenomethionine. Ascorbates serve a variety of other functions that promote cardiovascular health including: catalyzing the actions of phenylalanine and tyrosine; converting the-inactive form of folic acid to the active folinic acid; protecting B-vitamin complex, as well as vitamins A and E against oxidation; stimulating calcium metabolism; and, stimulating the activity of oxidative enzymes to protect against oxidation.

The novel compositions of the present invention may also comprise zinc, preferably in an amount in the range of about 4 mg to about 6 mg. Zinc is present in most tissues, with the greatest amounts occurring in the liver, voluntary muscle and bone. Zinc is a constituent of insulin and is required in more than 300 enzymes in the body. It plays an important role in nucleic acid metabolism and may be found in the form of zinc orotate. A preferred embodiment of the composition of the present invention may comprise about 5 mg of zinc.

Both the calcium and zinc of the present invention preferably may be in the form of orotate. Orotate is a chelator that decreases incidence of necrosis in the heart muscle and increases the rate of regeneration of healthy cellular and fibrous connective tissue in the heart region, and enhances cardiac mass and work capacity by increasing nucleic acid content and protein synthesis.

The compositions of the present invention may also comprise vitamin E in amounts preferably ranging from about 16 IU to about 24 IU. Vitamin E is a known antioxidant that prevents saturated fatty acids and vitamin A from combining with harmful substances. It enhances cellular respiration by uniting with oxygen, preventing it from being converted into various oxides so that blood cells are free to carry oxygen to body parts. Vitamin E supplementation has been exhaustively examined with studies done in rabbits (Wildlund et al., 32 J. LIPID. RES. 55-62 (1991); Kleinveld et al., 14 ARTERIOSCLER. THROMB. 1386-91 (1994)), in monkeys (Verlangieri and Bush, 11 J AM. COLL., NUTR. 130-7 (1992)), in cell cultures (Jessup et al., 265 BIOCHEM. J. 399-405 (1990)), and in humans (Princen et al., 15 THROMB. VASC. BIOL. 325-33 (1995); Suzukawa et al., 14 J. AM. COLL. NUTR. 46-52 (1995); Dieber-Rotheneder et al., 32 J. LIPID RES. 1325-32 (1991)). Studies further show that the cormbination of vitamin C and vitamin E supplements was better than either alone in preventing heart disease (Losonczy et al., 64 AM. J. CLIN. NUTR. 190-6 (1996)). Vitamin E and selenium are known to be synergistic, each protecting the body against oxidative free radicals using both a common mechanism of action and their own specific modes of action. A preferred embodiment of the compositions of the present invention may comprise about 20 IU vitamin E.

The compositions of the present invention may comprise vitamin A, in amounts preferably ranging from about 2000 IU to about 3000 IU. Vitamin A is a lipophilic antioxidant found naturally in the body. Beta-carotene, which is converted into vitamin A in the body, has been demonstrated to reduce risk of major coronary events by 51% (Gaziano and Hennekens 7 CONT. INT. MED. 9-14 (1995)). Beta-carotene supplementation works best in combination with vitamin E and should not be supplemented alone (Knekt et al., 139 AM. J. EPIDEMIOL. 1180-9 (1994)). Vitamin A indirectly may be beneficial in oral chelation because it assists in selenium utilization. Vitamin A thus helps to protect the body tissues from the irrtating effects of stress, smoke, air pollution, and chemical exposure. A preferred embodiment of the compositions of the present invention may comprise about 2500 IU vitamin A.

In another embodiment, the compositions of the present invention may comprise alpha-lipoic acid in amounts preferably ranging from about 16 mg to about 24 mg. Alphalipoic acid, or lipoic acid, is an amphiphilic antioxidant meaning that it functions both in fat and water. Research suggests that lipoic acid may help regenerate other key antioxidants by replenishing their electrons, thereby restoring these antioxidants to an oxidized state. Lipoic acid was also found to be a metal chelator, providing antioxidant activity by chelating Fe²⁺ and Cu²⁺ (Biewenga et al., 29(3) GEN. PHARMACOL. 315-31 (1997)). Other studies have suggested that when the body is deficient in other antioxidants, alpha-lipoic acid is able to do the work (Packer et al., 19 FREE RADIC. BIOL. MED. 227-50 (1995); Podda et al., 204 BIOCHEM. BIOPHYS. RES. COMMUN. 98-104 (1994)). A preferred embodiment of the compositions of the present invention may comprise about 20 mg alpha-lipoic acid.

The compositions of the present invention may also comprise Allium sativum (garlic), preferably in extract form, in amounts preferably ranging from about 16 mg to about 24 mg. The most common preparations of garlic make use of the bulb of the plant in various preparations including dried, aged or deodorized garlic preparations, garlic powder and garlic oil. Its constituents include 17 of the 20 naturally ocurring amino acids; alanine, arginine, aspartic acid, cystine, glutamic acid, glycine, histidine, isoleucine, leucine, lysine, methionine, phenylalanine, serine, threonine, tryptophan, tyrosine, valine as well as ascorbic acid, calcium, magnesium, phosphorus, potassium, allicin and allimn. See KOCH ET AL., GARLIC: THE SCIENCE AND THERAPEUTIC APPLICATION OF ALLIUM SATIVUM L AND RELATED SPECIES (Williams & Wilkins) (1996); FULDER, THE GARLIC BOOK: NATURE'S POWERFUL HEALER (Avery Publishing Group) (1997); and HEINERMAN FROM PHARAOHS TO PHARMACISTS: THE HEALING BENEFITS OF GARLIC (Keats Publishing) (1994).

Alliin is one of the components of garlic. Garlic powder is standardized to contain 1.3% alllin. Alliin itself is not useful until it is converted to the active form, allicin, by the enzyme allinase (KOCH ET AL., supra). Importantly, allicin has been determined to inhibit the biosynthesis of cholesterol (Gebhardt et al., 1213 BIOCHEM. BIOPHYS. ACTA 57-62 (1994)). Garlic preparations have been shown to reduce plaque deposit buildup by 50% and to slow hardening of the arteries in animals (Efendi et al., 132(1) ATHEROSCLEROSIS 37-42 (1997); SCHULZ ET AL., RATIONAL PHYTOTHERAPY 112 (Springer-Verlag) (1998)). Therefore, the administration of a composition of the present invention that contains garlic aids in removing buildup from and strengthening the arterial walls. In a preferred embodiment of the present invention the compositions comprise about 20 mg Allium sativum extract/powder. In another preferred embodiment, the Allium sativum extract/powder comprises at least about 0.8 percent allicin.

The compositions of the present invention may comprise Medicago sativa extract (alfalfa), preferably in amounts ranging from 16 mg to about 24 mg. The most common preparations of alfalfa include the whole plant and alfalfa root. Its constituents include triterpenoid saponins, phytosterols, coumarin, flavonoids, and alkaloids as well as calcium, carotene, chlorophyll, vitamin K, tannins, and trace elements (magnesium, phosphorus, and potassium). See Berrang, 13 PHYTOCHEMISTRY 2253 (1974), Gestetner, 10 PHYTOCHEMISTRY 2221 (1974), and Larher et al., 29(2/3) PLANT SCI. LETT. 315 (1983).

Triterpenoid saponins, also known as dietary saponins or alfalfa saponins, are the active principle of alfalfa. Triterpenoid saponins have been determined to lower LDL cholesterol levels in rabbits and monkeys. See, Reilly 1(1) J. NATUROPATHIC MED. 62-5 (1990); Cookson et al., 7 J ATHERSCLER. RES. 69 (1967); Malinow et al., 29 STEROIDS 10S (1977). The best known of the isolated triterpenoid saponins are the medicagenic acid glycosides which makes up 50-70% of the total aglycones received from the hydrolysis of alfalfa aerial parts or roots (Tava et al., 4 PHYTOCHEM. ANAL. 269 (1993)). Other aglycones include the hederagenin glycosides, found mostly in alfalfa roots, zanhic acid glycosides, found mostly in alfalfa plant tops, and soyasapogenol B and its dehydro glycosides, also found mostly in alfalfa plant tops. Both Procter & Gamble (see U.S. Pat. No. 5,591,836) and Pfizer, Inc. (see PCT/U.S.93/04092 published as WO 94/00480) have developed and patented semisynthetic saponins which have been demonstrated to inhibit cholesterol absorption. Some earlier studies show that phytosterols exert a greater effect on the reduction of total cholesterol than the saponins. These plant sterols have limited absorption in the intestines and compete with cholesterol for binding sites. The overall effect is a decrease in total cholesterol absorption (Dixit et al., 29 J PHYSIOL. PHARMACOL. 47 (1985); Peterson et al., 47 J. NUTR. 57 (1950)). Therefore, the administration of a composition of the present invention that contains alfalfa aids in LDL cholesterol lowering. In a preferred embodiment of the present invention, the compositions comprise about 20 mg Medicago sativa extract. In another preferred embodiment, the Medicago sativa extract comprises at least 0.06 percent saponins.

The compositions of the present invention may comprise Chondrus crispus extract (Irish moss), preferably in amounts ranging from about 24 mg to about 36 mg. The most common preparations of Irish moss include the dried and bleached thallus of this Irish seaweed better known as carrageen. Its constituents include flavodoxins, carrageenans and mineral salts (iodides and bromides) as well as calcium, magnesium, phosphorus, potassium and sodium. See CHAPMAN ET AL., SEAWEEDS AND THEIR USES (Chapmann and Hall) (1980). In a preferred embodiment of the present invention the compositions comprise about 30 mg Chondrus crispus extract.

Carrageenans are the active principle of Irish moss. A study done on the effects of carrageenans on heart disease shows that rats feeding on carageenans saw a lowering effect on serum cholesterol (Shiau and Huang, 35 NUTR. REP. INT. 479-86 (1987)). Depending on when the Irish moss is harvested, the carrageenan may comprise different mixtures of three family of carrageenans, kappa, iota and lambda, differing in the number and position of the ester sulfate groups on repeating galactose units.

As discussed in detail above, in one aspect of the invention, the novel compositions of the present invention preferably comprise: calcium; magnesium; selenium; manganese; zinc; potassium; vitamin E; vitamin A; alpha-lipoic acid; Allium sativum extract; Medicago sativa extract; Chondrus crispus extract; L-cysteine; L-glutamic acid; glycine; and glutathione.

Another aspect of the present invention relates to compositions and methods for promoting a healthy cardiovascular system and encouraging healthy blood flow by, e.g., removing toxic buildup from the arterial walls and dilating and strengthening the arterial walls. These methods comprise administering a composition comprising: calcium; magnesium; selenium; manganese; zinc; potassium; vitamin E; vitamin A; alpha-lipoic acid; Allium sativum extract; Medicago sativa extract; Chondrus crispus extract; L-cysteine; L-glutamic acid; glycine; and glutathione.

In a preferred aspect of the invention, a composition of the present invention is administered to reduce or control blood pressure in persons having a systolic pressure over about 130 mm Hg and/or a diastolic pressure over about 85 mm Hg. In another embodiment of the invention, the compositions are administered to reduce or control blood cholesterol levels in persons having a total cholesterol of about 240 mg/dL (5.95 mmol/L) or higher. In another embodiment of the invention, the compositions are administered to reduce levels of LDL-cholesterol in persons with a LDL-cholesterol of about 130 mg/dL (3.41 mmol/L) or higher. In another embodiment, a composition of the present invention is administered to raise levels of HDL to persons with a HDL-cholesterol of about 35 mg/dL (1.04 mmol/L) or lower to reduce the risk of atherosclerosis associated with low HDL levels. The compositions and methods of the present invention may also be utilized to improve or maintain vascular health in specific organ systems including the cardiovascular system, the cereberovascular system, the peripheral vascular system, and the intestinal vascular system.

In a preferred embodiment of the invention, the present composition is formulated for oral administration. Any dosage form may be employed for providing the patient with a dosage of the present compositions. Dosage forms include tablets, capsules, dispersions, suspensions, solutions, capsules, transdermal delivery systems, etc. Tablets and capsules represent the most advantageous oral dosage unit form. Any method known to those of ordinary skill in the art may be used to prepare capsules, tablets, or other dosage formulations. Tablets or capsules can be coated by methods well known to those of ordinary skill in the art.

According to one aspect of the invention a composition is provided comprising a pharmaceutically acceptable combination of the composition and at least one carrier. Pharmaceutically acceptable carriers for inclusion into the present compositions include carriers most suitable for combination with lipid-based drugs such as diluents, excipients and the like which enhance its oral administration. Suitable carriers include, but are not limited to, sugars, starches, cellulose and derivatives thereof, wetting agents, lubricants such as sodium lauryl sulfate, stabilizers, tableting agents, anti-oxidants, preservatives, coloring agents and flavoring agents. Pharmaceutically acceptable carriers include binding agents such as pregelatinized maize starch, polyvinylpryrrolidone or hydroxypropyl methycellulose; binders or fillers such as lactose, pentosan, microcrystalline cellulose or calcium hydrogen phosphate; lubricants such as magnesium stearate, talc or silica; disintegrants such as potato starch or sodium starch; or wetting agents such as sodium lauryl sulfate. Reference may be made to REMINGTON'S PHARMACEUTICAL SCIENCES, 17TH ED., 1985, for other carriers that would be suitable for combination with the present compositions. As will be appreciated, the pharmaceutical carriers used to prepare compositions in accordance with the present invention will depend on the administrable form to be used.

According to an additional embodiment, the compositions of the present invention may be admixed by conventional methods and may be administered by an alternative route such as suppository, spray, liquid, powder, liposome, dermal patch, and inhalant. These methods are well known to those skilled in the art. For example, liposomes may be formulated according to methods such as those of U.S. Pat. Nos. 5,853,755, 4,235,871, or 4,708,861 (liposome-gel combination). Sublingual and transdermal methods are also well known to those skilled in the art, e.g., U.S. Pat. No. 5,922,342 describes a sublingual formulation and U.S. Pat. No. 4,997,655 describes a transdermal administration method.

Another embodiment of the invention involves administering the composition of the present invention to a human in one or more tablets as a dietary supplement. In yet another embodiment of the invention, the composition is administered to a human as a pharmaceutical composition.

The administration of the composition would be in accordance with a predetermined regimen, which would be at least three times daily and over an extended period of time as a chronic treatment, and could last for one year or more, including the life of the patient. The dosage administered will depend upon the frequency of the administration, the blood level desired, other concurrent therapeutic treatments, the severity of the condition, whether the t treatment is for prophylaxis or therapy, the age of the patient, the levels of LDL-cholesterol and HDL-cholesterol in the patient, and the like.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description.

### EXAMPLE 1

Composition 1

A composition of the following formulation was prepared in tablet form by standard methods known to those skilled in the art:

| | | |
|---|---|---|
| Calcium (as orotate and ascorbate) | 30 | mg |
| Magnesium (as succinate) | 30 | mg |
| Selenium (as selenomethionine) | 20 | mcg |
| Manganese (as ascorbate) | 0.5 | mg |
| Zinc (as orotate) | 5 | mg |
| Potassium (as citrate) | 30 | mg |
| Vitamin E | 20 | IU |
| Vitamin A | 2500 | IU |
| Alpha-lipoic acid | 20 | mg |
| Alllium sativum extract | 20 | mg |
| (bulb, 0.8% allicin) | | |
| Medicago sativa extract | 20 | mg |
| Chondrus crispus extract | 30 | mg |
| L-cysteine | 20 | mg |
| L-glutamic acid | 20 | mg |
| Glycine | 20 | mg |
| Glutathione | 15 | mg |

Three to five tablets per day is the recommended dosage for an average weight adult human (70-kg).

Clinical Studies:

A study of the effect of a dietary supplement comprising, calcium, magnesium, selenium, manganese, zinc, potassium, vitamin E, vitamin A, alpha-lipoic acid; Allium sativum extract, Medicago sativa extract, Chondrus crispus extract, L-cysteine, L-glutamic acid, glycine, and glutathione, on blood pressure, HDL cholesterol level, LDL cholesterol level and plasma lipid peroxide level is conducted over a six-month period. Plasma lipid peroxide levels were determined according to the procedure described by Gorog et al., 111(1) ATHEROSCLEROSIS 47-53 (1994). A statistical analysis is performed to compare the resulting blood pressure, HDL cholesterol, LDL cholesterol and plasma lipid peroxide levels and a control (placebo) group to measure the improvement in cardiovascular health from administration of the test preparation.

Sixty adult individuals are selected for inclusion in the statistical study. Two weeks prior to the start of the study, each subject completes a one-week dietary intake record and is interviewed by a Registered Dietitian to calculate each individual's daily energy requirement. Baseline blood pressure, HDL cholesterol, LDL cholesterol and plasma lipid peroxide levels are determined with blood samples drawn on two separate days. Subjects are randomly assigned to one of two treatment groups: the group receiving the test tablets (Composition 1) or the group receiving matching placebo tablets. Both groups continue on their normal diet and incorporate three tablets of either the Composition I or placebo in their daily diet.

The effects of supplementing the diet with the above composition are evaluated for blood pressure, HDL cholesterol, LDL cholesterol and plasma lipid peroxide levels using multiple linear regression analysis and a standard students t-test. In each analysis; the baseline value of the outcome variable is included in the model as a covariant. Treatment by covariant interaction effects is tested by the method outlined by Weigel and Narvaez, 12 CONTROLLED CLINICAL TRIALS 378-394 (1991). In the absence of significant interaction effects, the interaction terms are removed from the model. Regression model assumptions of normality and homogeneity residual variance are evaluated by inspection of plots of residuals M- versus plots of predicted values. Temporal outset of effects is detected sequentially by testing for the presence of significant treatment effects at 18, 12, and 6 weeks, proceeding to the earlier time in sequence only when significant effects have been identified at each later time period. In addition, one-way analysis of variance is compared only when differences exist between groups concerning nutrient intake, physical activity, and body mass index at X each time point. Changes from the baseline within each group are evaluated using paired t-tests. Additionally, analysis of variance is performed on all baseline measurements and measurable subject characteristics to assess homogeneity between groups. All statistical procedures are conducted using the Statistical Analysis System (SAS Institute Inc., Cary, N.C.) using an alpha level of 0.05 for all statistical tests.

A statistically significant decrease in blood pressure, LDL cholesterol and plasma lipid peroxide levels and increase in HDL cholesterol level are observed in the treated subjects upon completion of the study, but not in the control subjects.

### EXAMPLE 2

A study of the effect of the composition comprising, calcium, magnesium, selenium, manganese, zinc, potassium, vitamin E, vitamin A, alpha-lipoic acid, Allium sativum extract, Medicago saliva extract, Chondrus crispus extract, L-cysteine, L-glutarnic acid, glycine, and glutathione, on blood pressure, HDL cholesterol, LDL cholesterol and plasma lipid peroxide levels, is conducted over a nine-week period. Plasma lipid peroxide levels were determined according to the procedure described by Gorog et al., supra. A statistical analysis is performed to compare blood pressure, HDL cholesterol, LDL cholesterol and plasma lipid peroxide levels of a test group and a control (placebo) group to determine if a significant improvement in blood pressure, HDL cholesterol, LDL cholesterol and plasma lipid peroxide levels results from administration of the test preparation.

Sixty adult individuals with no history or symptoms of heart disease are selected for inclusion in the statistical study. Each patient was held in a clinical setting for a total of nine weeks. A total of three isocaloric diets were imposed, and each patient held on each diet for three weeks. The components of each diet may vary as long as lipid levels in the diet were maintained. The three diets are as follows:

| American Heart Association Diet II | |
|---|---|
| Fat | 25% |
| Cholesterol | 80 mg/1000 kCal |
| Polyunsaturated/saturated fat | 1.5 |
| Average American Diet | |
| Fat | 43% |
| Cholesterol | 200 mg/1000 kCal |
| Polyunsaturated/saturated fat | 0.34 |
| Combination Diet | |
| Fat | 43% |
| Cholesterol | 80 mg/1000 kCal |
| Polyunsaturated/saturated fat | 0.34 |

Subjects are randomly assigned to one of two treatment groups: the group receiving the test tablets (Composition 1) or the group receiving matching placebo tablets. Both groups continue on the lipid-controlled diet and incorporate three tablets of either the Composition 1 or placebo in their daily diet. At the end of each three-week period, blood pressure, HDL cholesterol, LDL cholesterol and plasma lipid peroxide levels are determined.

The effects of supplementing the diet with the above composition are evaluated for blood pressure, HDL cholesterol, LDL cholesterol and plasma lipid peroxide levels using multiple linear regression analysis and a standard students t-test. In each analysis, the baseline value of the outcome variable is included in the model as a covariant. Treatment by covariant interaction effects is tested by the method outlined by Weigel and Narvaez, supra. In the absence of significant interaction effects, the interaction terms are removed from the model. Regression model assumptions of normality and homogeneity residual variance are evaluated by inspection of plots of residuals versus plots of predicted values. Temporal outset of effects is detected sequentially by testing for the presence of significant treatment effects at 18, 12, and 6 weeks, proceeding to the earlier time in sequence only when significant effects have been identified at each later time period. In addition, one-way analysis of variance is compared only when differences exist between groups concerning nutrient intake, physical activity, and body mass index at each time point. Changes from the baseline within each group are evaluated using paired t-tests. Additionally, analysis of variance is performed on all baseline measurements and measurable subject characteristics to assess homogeneity between groups. All statistical procedures are conducted using the Statistical Analysis System (SAS Institute Inc., Cary, N.C.) using an alpha level of 0.05 for all statistical tests.

A statistically significant decrease in blood pressure, LDL cholesterol and plasma lipid peroxide levels and-increase in HDL cholesterol level are observed in the treated subjects upon completion of the study, but not in the control subjects.

The invention has been described in detail with particular reference to preferred embodiment thereof. However, it will be appreciated that those skilled in the art, upon consideration of this disclosure may make variations and modifications within the spirit and scope of the invention.

The disclosure of all publications cited above are expressly incorporated by reference in their entireties to the same extent as if each were incorporated by reference individually.

I claim:

1. A composition comprising: calcium; magnesium; selenium; manganese; zinc; potassium; vitamin E; vitamin A; alpha-lipoic acid; Allium sativum extract; Medicago sativa extract; Chondrus crispus extract; L-cysteine; L-glutamic acid; glycine; and glutathione.

2. The composition of claim 1, wherein said calcium comprises calcium orotate and calcium abscorbate.

3. The composition of claim 1, wherein said magnesium comprises magnesium succinate.

4. The composition of claim 1, wherein said selenium comprises selenomethionine.

5. The composition of claim 1, wherein said manganese comprises manganese ascorbate.

6. The composition of claim 1, wherein said zinc comprises zinc orotate.

7. The composition of claim 1, wherein said potassium comprises potassium citrate.

8. The composition of claim 1, wherein said Allium sativum extract comprises about 0.8% allicin.

9. The composition of claim 1, wherein said Medicago sativa extract comprises triterpeniod saponins.

10. The composition of claim 1, wherein said Chondrus crispus extract comprises carrageenans.

11. The composition of claim 1, wherein said calcium is present in an amount in the range of about 24 mg to about 36 mg.

12. The composition of claim 1, wherein said magnesium is present in an amount in the range of about 24 mg to about 36 mg.

13. The composition of claim 1, wherein said selenium is present in an amount in the range of about 16 mcg to about 24 mcg.

14. The composition of claim 1, wherein said manganese is present in an amount in the range of about 0.3 mg to about 0.7 mg.

15. The composition of claim 1, wherein said zinc is present in an amount in the range of about 4 mg to about 6 mg.

16. The composition of claim 1, wherein said potassium is present in an amount in the range of about 24 mg to about 36 mg.

17. The composition of claim 1, wherein said vitamin E is present in an amount in the range of about 16 IU to about 24 IU.

18. The composition of claim 1, wherein said vitamin A is present in an amount in the range of about 2000 IU to about 3000 IU.

19. The composition of claim 1, wherein said alpha-lipoic is present in an amount in the range of 16 mg to about 24 mg.

20. The composition of claim 1, wherein said Allium sativum extract is present in an amount in the range of about 16 mg to about 24 mg.

21. The composition of claim 1, wherein said Medicago sativa extract is present in an amount in the range of about 16 mg to about 24 mg.

22. The composition of claim 1, wherein said Chondrus crispus extract is present in an amount in the range of about 24 mg to about 36 mg.

23. The composition of claim 1, wherein said L-cysteine is present in an amount in the range of about 16 mg to about 24 mg.

24. The composition of claim 1, wherein said L-glutamic acid is present in an amount in the range of about 16 mg to about 24 mg.

25. The composition of claim 1, wherein said glycine is present in an amount in the range of about 16 mg to about 24 mg.

26. The composition of claim 1, wherein said glutathione is present in an amount in the range of about 12 mg to about 18 mg.

27. The composition of claim 1, wherein said composition comprises: 30 mg calcium; 30 mg magnesium; 20 mcg selenium; 0.5 mg manganese; 5 mg zinc; 30 mg potassium; 20 IU vitamin E; 2500 IU vitamin A; 20 mg alpha-lipoic acid; 20 mg Allium sativum extract (0.8% allicin); 20 mg Medicago sativa extract; 30 mg Chondrus crispus extract; 20 mg L-cysteine; 20 mg L-glutamic acid; 20 mg glycine; and 20 mg glutathione.

28. A method of promoting a healthy cardiovascular system comprising the step of administering to said mammal a composition comprising: calcium; magnesium; selenium; manganese; zinc; potassium; vitamin E; vitamin A; alpha-lipoic acid; Allium sativum extract; Medicago sativa extract; Chondrus crispus extract; L-cysteine; L-glutamic acid; glycine; and glutathione.

29. The method of claim 28, wherein said calcium comprises calcium orotate and calcium abscorbate.

30. The method of claim 28, wherein said magnesium comprises magnesium succinate.

31. The method of claim 28, wherein said selenium comprises selenomethionine.

32. The method of claim 28, wherein said manganese comprises manganese ascorbate.

33. The method of claim 28, wherein said zinc comprises zinc orotate.

34. The method of claim 28, wherein said potassium comprises potassium citrate.

35. The method of claim 28, wherein said Allium sativum extract comprises about 0.8% allicin.

36. The method of claim 28, wherein said Medicago sativa extract comprises triterpenoid saponins.

37. The method of claim 28, wherein said Chondrus crispus extract comprises carrageenans.

38. The method of claim 28, wherein said calcium is present in an amount in the range of about 24 mg to about 36 mg.

39. The method of claim 28, wherein said magnesium is present in an amount in the range of about 24 mg to about 36 mg.

40. The method of claim 28, wherein said selenium is present in an amount in the range of about 16 mcg to about 24 mcg.

41. The method of claim 28, wherein said manganese is present in an amount in the range of about 0.3 mg to about 0.7 mg.

42. The method of claim 28, wherein said zinc is present in an amount in the range of about 4 mg to about 6 mg.

43. The method of claim 28, wherein said potassium is present in an amount in the range of about 24 mg to about 36 mg.

44. The method of claim 28, wherein said vitamin E is present in an amount in the range of about 16 IU to about 24 IU.

45. The method of claim 28, wherein said vitamin A is present in an amount in-the range of about 2000 IU to about 3000 IU.

46. The method of claim 28, wherein said alpha-lipoic is present in an amount in the range of 16 mg to about 24 mg.

47. The method of claim 28, wherein said Allium sativum extract is present in an amount in the range of about 16 mg to about 24 Mg.

48. The method of claim 28, wherein said Medicago sativa extract is present in an amount in the range of about 16 mg to about 24 mg.

49. The method of claim 28, wherein said Chondrus crispus extract is present in an amount in the range of about 24 mg to about 36 mg.

50. The method of claim 28, wherein said L-cysteine is present in an amount in the range of about 16 mg to about 24 mg.

51. The method of claim 28, wherein said, L-glutamic acid is present in an amount in the range of about 16 mg to about 24 mg.

52. The method of claim 28, wherein said glycine is present in an amount in the range of about 16 mg to about 24 mg.

53. The method of claim 28, wherein said glutathione is present in an amount in the range of about 12 mg to about 18 mg.

54. The method of claim 28, wherein said composition comprises: 30 mg calcium; 30 mg magnesium; 20 mcg selenium; 0.5 mg manganese; 5 mg zinc; 30 mg potassium; 20 IU vitamin E; 2500 IU vitamin A; 20 mg alpha-lipoic acid; 20 mg Allium sativum extract (0.8% allicin); 20 mg Medicago saliva extract; 30 mg Chondrus crispus extract; 20 mg L-cysteine; 20 mg L-glutamic acid; 20 mg glycine; and 20 mg glutathione.

55. The method of claim 28, further comprising a pharmaceutically acceptable carrier.

56. The method of claim 28, wherein said composition is administered 3 to 5 times daily.

57. The method of claim 28, wherein said composition is administered orally.

58. The method of claim 28, wherein said composition is provided as a liquid in the form of an emulsion.

59. The method of claim 28, wherein said composition is provided in capsular form.

60. The method of claim 28, wherein said composition is provided in tablet form.

61. The method of claim 28, wherein said masimal is a human.

62. The method of claim 61, wherein said promoting a healthy cardiovascular system comprise the step of reducing high blood pressure.

63. The method of claim 62, wherein said reducing high blood pressure reduces blood pressure in persons having a systolic pressure over 130 mm Hg and/or a diastolic pressure over 85 mm Hg.

64. The method of claim 61, wherein said promoting a healthy cardiovascular system comprise the step of increasing HDL cholesterol level.

65. The method of claim 64, wherein said increasing HDL cholesterol level increases HDL cholesterol level in persons having a HDL cholesterol level of over 35 mg/dL (1.04 mmol/L).

66. The method of claim 61, wherein said promoting a healthy cardiovascular system comprise the step of reducing total cholesterol level.

67. The method of 66, wherein said reducing total cholesterol level reduces blood cholesterol level in persons having a total cholesterol of 240 mg/dL (5.95 mmol/L) or higher.

68. The method of claim 61, wherein said promoting a healthy cardiovascular system comprise the step of reducing LDL cholesterol level.

69. The method of claim 68, wherein said reducing of LDL cholesterol level reduces LDL cholesterol in persons with a LDL-cholesterol level of 130 mg/dL (3.41 mmol/L) or higher.

70. The method of claim 61, wherein said promoting a healthy cardiovascular system comprise the step of maintaining or reducing lipid peroxide levels.

71. A method of enhancing blood flow comprising the step of administering a composition comprising: calcium; magnesium; selenium; manganese; zinc; potassium; vitamin E; vitamin A; alpha-lipoic acid; Allium sativum extract; Medicago sativa extract; Chondrus crispus extract; L-cysteine; L-glutamic acid; glycine; and glutathione.

72. The method of claim 71, wherein said calcium comprises calcium orotate and calcium abscorbate.

73. The method of claim 71, wherein said magnesium comprises magnesium succinate.

74. The method of claim 71, wherein said selenium comprises selenomethionine.

75. The method of claim 71, wherein said manganese comprises manganese ascorbate.

76. The method of claim 71, wherein said zinc comprises zinc orotate.

77. The method of claim 71, wherein said potassium comprises potassium citrate.

78. The method of claim 71, wherein said Allium sativum extract comprises about 0.8% allicin.

79. The method of claim 71, wherein said Medicago sativa extract comprises triterpenoid saponins.

80. The method of claim 71, wherein said Chondrus crispus extract comprises carrageenans.

81. The method of claim 71, wherein said calcium is present in an amount in the range of about 24 mg to about 36 mg.

82. The method of claim 71, wherein said magnesium is present in an amount in the range of about 24 mg to about 36 mg.

83. The method of claim 71, wherein said selenium is present in an amount in the range of about 16 mcg to about 24 mcg.

84. The method of claim 71, wherein said manganese is present in an amount in the range of about 0.3 mg to about 0.7 mg.

85. The method of claim 71, wherein said zinc is present in an amount in the range of about 4 mg to about 6 mg.

86. The method of claim 71, wherein said potassium is present in an amount in the range of about 24 mg to about 36 mg.

87. The method of claim 71, wherein said vitamin E is present in an amount in the range of about 16 IU to about 24 IU.

88. The method of claim 71, wherein said vitamin A is present in an amount in the range of about 2000 IU to about 3000 IU.

89. The method of claim 71, wherein said alpha-lipoic is present in an amount in the range of 16 mg to about 24 mg.

90. The method of claim 71, wherein said Allium sativum extract is present in an amount in the range of about 16 mg to about 24 mg.

91. The method of claim 71, wherein said Medicago sativa extract is present in an amount in the range of about 16 mg to about 24 mg.

92. The method of claim 71, wherein said Chondrus crispus extract is present in an amount in the range of about 24 mg to about 36 mg.

93. The method of claim 71, wherein said L-cysteine is present in an amount in the range of about 16 mg to about 24 mg.

94. The method of claim 71, wherein said L-glutamic acid is present in an amount in the range of about 16 mg to about 24 mg.

95. The method of claim 71, wherein said glycine is present in an amount in the range of about 16 mg to about 24 mg.

96. The method of claim 71, wherein said glutathione is present in an amount in the range of about 12 mg to about 18 mg.

97. The method of claim 71, wherein said composition comprises: 30 mg calcium; 30 mg magnesium; 20 mcg selenium; 0.5 mg manganese; 5 mg zinc; 30 mg potassium; 20 IU vitamin E; 2500 IU vitamin A; 20 mg alpha-lipoic acid; 20 mg Allium sativum extract (0.8% allicin); 20 mg Medicago sativa extract; 30 mg Chondrus crispus extract; 20 mg L-cysteine; 20 mg L-glutamic acid; 20 mg glycine; and 20 mg glutathione.

98. The method of claim 71, further comprising a pharmaceutically acceptable carrier.

99. The method of claim 71, wherein said composition is administered 3 to 5 times daily.

100. The method of claim 71, wherein said composition is administered orally.

101. The method of claim 71, wherein said composition is provided as a liquid in the form of an emulsion.

102. The method of claim 71, wherein said composition is provided in capsular form.

103. The method of claim 71, wherein said composition is provided in tablet form.

104. The method of claim 71, wherein said mammal is a human.

105. The method of claim 104, wherein said promoting a healthy cardiovascular system comprise the step of reducing high blood pressure.

106. The method of claim 105, wherein said reducing high blood pressure reduces blood pressure in persons having a systolic pressure over 130 mm Hg and/or a diastolic pressure over 85 mm Hg.

107. The method of claim 104, wherein said promoting a healthy cardiovascular system comprise the step of increasing HDL cholesterol level.

108. The method of claim 107, wherein said increasing HDL cholesterol level increases HDL cholesterol level in persons having a HDL cholesterol level of over 35 mg/dL (1.04 mmol/L).

109. The method of claim 104, wherein said promoting a healthy cardiovascular system comprise the step of reducing total cholesterol level.

110. The method of 109, wherein said reducing total cholesterol level reduces blood cholesterol level in persons having a total cholesterol of 240 mg/dL (5.95 mmol/L) or higher.

111. The method of claim 104, wherein said promoting a healthy cardiovascular system comprise the step of reducing LDL cholesterol level.

112. The method of claim 111, wherein said reducing of LDL cholesterol level reduces LDL cholesterol in persons with a LDL-cholesterol level of 130 mg/dL (3.41 mmol/L) or higher.

113. The method of claim 104, wherein said promoting a healthy cardiovascular system comprise the step of maintaining or reducing lipid peroxide levels.
